(19) United States
(12) Patent Application Publication
(10) Pub. No.: US 2013/0059814 A1
(43) Pub. Date: Mar. 7, 2013

(54) ANTI-INFLAMMATORY DERMATOLOGICAL COMPOSITION COMPRISING CORTICOSTEROIDS AND HYALURONATE FRAGMENTS, AND USES THEREOF

(71) Applicants: Christine Chaumont, Toulouse (FR); Jean-Hilaire Saurat, Geneve (FR); Gürkan Kaya, Geneve (CH)

(72) Inventors: Christine Chaumont, Toulouse (FR); Jean-Hilaire Saurat, Geneve (FR); Gürkan Kaya, Geneve (CH)

(21) Appl. No.: 13/603,146

(22) Filed: Sep. 4, 2012

Related U.S. Application Data
(62) Division of application No. 12/669,336, filed on Jan. 15, 2010, now abandoned.

(30) Foreign Application Priority Data
Jul. 16, 2007 (FR) 0756515

(51) Int. Cl. A61K 31/728; A61P 17/00; A61P 29/00

(52) U.S. Cl. 514/54

(57) ABSTRACT

Anti-inflammatory dermatological composition for topical administration, characterized in that it comprises 0.005% to 0.1%, preferably 0.01% to 0.05% by weight of a corticosteroid and 0.1% to 1%, preferably 0.5% to 1% by weight of hyaluronate fragments with an average molecular weight of between 20 and 500 kDa, preferably between 20 and 375 kDa, more preferentially between 20 and 150 kDa.

The present application is a 37 C.F.R. §1.53(b) divisional of, and claims priority to, U.S. application Ser. No. 12/669,336, filed Jan. 15, 2010. Application Ser. No. 12/669,336 is the national phase under U.S.C. §371 of International Application No. PCT/EP2008/059021, filed on July 10, 2008. Priority is also claimed to French Application No. 0756515 filed on Jul. 16, 2007. The entire contents of each of these applications is hereby incorporated by reference.

The present invention relates to a dermatological composition comprising corticosteroids and hyaluronate fragments as well as to their uses.

Hyaluronate (HA) is the major component of the extracellular matrix and is found in significant amounts in the skin. HA is a linear glycosaminoglycan non-sulfate consisting of recurrent units of D-glucuronic acid and N-acetyl-D-glucosamine (Tammi R., Agren U M., Tuhkanen A L., Tammi M. Hyaluronan metabolism in skin. Prog. Histochem. & Cytochem. 29: 1-81, 1994).

In normal skin, HA is essentially synthesized by dermal fibroblasts and epidermal keratinocytes (Tammi R., Agren U M., Tuhkanen A L., Tammi M. Hyaluronan metabolism in skin. Prog. Histochem. & Cytochem. 29: 1-81, 1994). By its residues bearing a negative charge, HA plays the role of a water pump with which visco-elasticity of skin may be maintained. HA has a main role in controlling diffusion of foodstuffs, hormones, vitamins, and inorganic salts of the connective tissue and in cleaning metabolic waste which may induce inflammatory reactions. With age, the amount of HA and its polymerization degree decrease, resulting in a reduction of the amount of water retained in the connective tissue. Skin then undergoes an ageing process which results in an increase of fibrosis and to a lowering of the elastic fiber content.

In normal skin, HA exists as a polymer of high molecular weight (600-1,000 kDa). Physiological degradation of HA in the skin is accomplished by (i) internalization by keratinocytes via CD44 and (ii) intracellular fragmentation into fragments of smaller size by hyaluronidases. The fragmented HA is released by the keratinocytes, passes the basal membrane and is directly released in the lymphatic vessels (Tammi R., Agren U M., Tuhkanen A L., Tammi M. Hyaluronan metabolism in skin. Prog. Histochem. & Cytochem. 29: 1-81, 1994).

Under inflammatory conditions, the accumulation of low molecular weight forms of HA has been demonstrated. During the inflammation, thrombocytic chemotactic factors such as fibrins stimulate the inflow and activation of fibroblasts which degrade HA by secretion of hyaluronidase resulting in high tissue concentrations of small fragments of HA. The generation of these small HA fragments is also accomplished by a variety of other mechanisms such as depolymerization by oxygen-reactive species released by granulocytes or in skin irradiated by ultraviolet radiation, or the de novo synthesis of fragments with low molecular weights. Several studies have suggested that high and low molecular weight HA may have different biological effects on cells and tissues (Mckee C M., Penno M B., Cowman M., Burdick M D., Strieter R M. I., Bao C I, Noble P W. Hyaluronan (HA) fragments induce chemokine gene expression in alveolar macrophages. The role of HA size and CD44. J. clin Invest. 98:2403-2143, 1996; Termeer C C., Hennies I., Voith U., Ahrens T., Weiss J M., Prehm P., Simon J C., Oligosaccharides of hyaluronan are potent activators of dendritic cells. J. Immunol. 165:1863-1870, 2000; Fitzgerald K A., Bowie A G., Skeffington B S., O'Neill L A., Ras, protein kinase C zeta, and I kappa B kinases 1 and 2 are downstream effectors of CD44 during the activation of NF-kappa B by hyaluronic acid fragments in T-24 carcinoma cells. J. Immunol 164: 2053-2063, 2000).

It has been demonstrated (FR 04 00826) that non-sulfated HA hydrolyzed into fragments with molecular weights comprised between 50 and 750 kDa, has biological activity on the skin, notably an increase in epidermis renewal, in the expression of epidermal CD44 and in extracellular matrix deposition, which is amplified when these fragments are associated with a retinoid.

CD44, the main receptor of HA, is a polymorphic transmembrane glycoprotein which has several isoforms generated by alternating splicing and post-translational modifications. It was demonstrated that two major functions of CD44 in murine skin are (i) regulation of keratinocyte proliferation in response to extracellular stimuli and (ii) the maintaining of local homeostasis of HA (Kaya G., Rodriguez L., Jorcano J L., Vassalli P., Stamenkovic I. Selective suppression of CD44 in keratinocytes of mice bearing an antisense CD44 transgene driven by a tissue-specific promoter disrupts hyaluronate metabolism in the skin and impairs keratinocyte proliferation. Genes, Dev. 11:996-1007, 1997). A reduction of the expression of epidermal CD44 in patients affected with sclero-atrophic lichen has also been observed. This reduction is potentially responsible for dermal deposition of HA and of epidermal atrophy in this disease (Kaya G., Augsburger E., Stamenkovic L., Saurat J H., Decrease in epidermal CD44 expression as a potential mechanism for abnormal hyaluronate accumulation in superficial dermis in lichen sclerosus and atrophicus. J. Invest. Dermatol. 115:1054-1058, 2000). It was recently demonstrated (i) that the in vitro and in vivo proliferative response of keratinocytes induced by HA fragments of intermediate size follows a CD44-dependent route and requires the presence of heparin-binding epidermal growth factor (HB-EGF), erbBI, and matrix metalloproteinases, and (ii) that the HA fragments of intermediate size may form a basis for the development of novel therapies for human skin atrophy (Kaya G., Tran C., Sorg O., Hotz R., Grand D., Carraux P., Didierjean L., Stamenkovic L., Saurat J.-H. Hyaluronate fragments reverse skin atrophy by a CD44-dependent mechanism. PloS Med. 3 (12): e493, 2006).

Moreover, it has recently been demonstrated that unlike fragments of small sizes (1-50 kDa) or large sizes (400-1,000 kDa), the HA fragments of intermediate size induce significant epidermal hyperplasia and keratinocyte proliferation, an increase in the expression of epidermal and dermal CD44 and HA as well as an alteration of the dermis structure and an increase in its cellularity in hairless SKH1 and DBA/1 mice. It has also finally been demonstrated that retinaldehyde prevents epidermal atrophy induced by a corticosteroid, clobetasol propionate, in hairless SKH1 mice (Kaya G., Tran C., Sorg O., Grand D., Hotz R., Carraux P., Didierjean L., Saurat J.-H. Prevention of corticosteroid-induced skin atrophy by retinaldehyde in mouse. JEADV 19: 124, 2005).

It had been observed earlier that HA fragments of an intermediate size allowed the repair of an already established atrophy, due to ageing, and worsened by the use of corticosteroids used via a systemic route in the long term (Kaya G., Tran C., Sorg O., Hotz R., Grand D., Carraux P., Didierjean L., Stamenkovic L., Saurat J.-H. Hyaluronate fragments reverse skin atrophy by a CD44-.dependent mechanism. PloS Med 3 (12): e493, 2006 and patent FR 04 00826).

The authors of the present invention have surprisingly discovered that it is possible to prevent the occurrence of skin atrophy by concomitant use of a corticosteroid and HA fragments with a molecular weight comprised between 20 and 500 kDa.

From the moment that they exert inhibition of the atrophying effect of corticosteroids, these HA fragments should also inhibit the other effects of corticosteroids, including the main sought therapeutic effect, i.e. the anti-inflammatory effect.

The authors of the present invention have surprisingly discovered that on the contrary, the concomitant use of a corticosteroid and of these HA fragments does not cancel out the anti-inflammatory effect of the corticosteroid.

Thus, the present invention somewhat allows dissociation of the therapeutic effect from the major secondary effect of topical corticosteroids. It therefore allows the use of a single topical preparation consisting of the association of HA fragments and of a corticosteroid.

By “dissociation of the therapeutic effect and of the secondary effect” is therefore meant the fact of reducing or even suppressing the atrophying properties of the corticosteroid while preserving its anti-inflammatory effect.

Further, and even in a more surprising way, potentialization of the anti-inflammatory therapeutic effect was even observed.

The invention therefore more specifically relates to an anti-inflammatory dermatological composition intended for topical administration, characterized in that it comprises 0.005-0.1%, preferably 0.01-0.05% by weight of a corticosteroid and 0.1-1%, preferably 0.5-1% by weight of hyaluronate fragments with an average molecular weight comprised between 20 and 500 kDa, preferably between 20 and 375 kDa, more preferentially between 20 and 150 kDa.

The intention is to describe as “anti-inflammatory”, in the sense of the present invention, the fact of inhibiting via a topical route, standard signs such as redness, oedema, vesicles, pain and pruritus, which are induced by a large number of pathologies at the skin, and which are attenuated by applying topical corticosteroids.

The intention is to describe as “potentializing”, in the sense of the present invention, the fact of avoiding the main secondary effect of the topical corticosteroid such as skin atrophy, while obtaining a better anti-inflammatory effect than the one which would be obtained with the same amount of topical corticosteroid alone, or the same anti-inflammatory effect as the one which would be obtained with a smaller amount of corticosteroid.

The hyaluronate fragments of the present invention may be obtained by heat treatment of fibers of sodium hyaluronate with a high molecular weight at a temperature above 100° C.

The fragments of hyaluronate may also be obtained by ultrasound treatment of fibers of sodium hyaluronate of high molecular weight, for 10-90 minutes, advantageously 45 minutes, at 400W and at 4° C., followed by filtering on a gel, advantageously on Sephacryl S-400 gel.

The composition according to the invention advantageously comprises 0.05% by weight of a corticosteroid and 0.5% by weight of hyaluronate fragments.

The composition according to the invention advantageously comprises 0.01% by weight of a corticosteroid and 1% by weight of hyaluronate fragments.

The corticosteroid may advantageously be selected from alclometasone dipropionate, amcinonide, beclometasone dipropionate, betamethasone benzoate, betamethasone dipropionate, betamethasone valerate, budesonide, clobetasol propionate, clobetasol butyrate, desonide, desoxymethasone, dexamethasone, diflorasone diacetate, diflucortolone valerate, flurandrenolone, fluprednidene acetate, fluocortolone, fluocortine butyl, fluocinonide, fluocinolone acetonide, fluclorolone acetonide, flumetasone pivalate, feudiline hydrochloride, flumetholone, halcinonide, hydrocortisone, hydrocortisone acetate, hydrocortisone butyrate, hydrocortisone valerate, methylprednisolone acetate, mometasone furoate, methylprednisolone, prednisolone, triamcinolone acetonide as well as mixtures thereof.

The corticosteroid is advantageously clobetasol propionate.

The object of the invention is also a pharmaceutical composition comprising a composition as defined above and one or more pharmaceutically acceptable excipients.

The pharmaceutical composition according to the invention advantageously comprises a pharmaceutically acceptable emollient base.

By “emollient base” is meant in the sense of the present invention any cosmetic product which contributes in releasing the tissues, soothing the inflammation and softening the skin.

The pharmaceutical composition according to the invention also advantageously comprises other dermatological acceptable excipients for its presentation as a cream, balm, gel, spray, ointment, lotion, film-forming solution, transdermal system, for example a patch, foam, shampoo.

The object of the invention is also a composition according to any of the preceding claims, as a drug, advantageously intended for treating inflammatory dermatoses, which are commonly listed as indications of topical corticosteroids, and more particularly those which are localized on fragile areas such as the face, areas where secondary effects of topical corticosteroids are particularly marked. Indeed, by the dissociation of the therapeutic effect and of the major secondary effect of topical corticosteroids on the one hand, and by the potentialization of the anti-inflammatory effect on the other hand, these fragile areas may be treated with less risk.

The object of the invention is also a combination product comprising a corticosteroid as a cream on the one hand, and hyaluronate fragments with an average molecular weight comprised between 200 and 500 kDa, advantageously between 20 and 375 kDa, more advantageously between 20 and 150 kDa, also as a cream, on the other hand, for a separate dermatological use, either simultaneous or spread out in time, in the therapy of inflammatory dermatoses.

The hyaluronate fragments may be obtained by either one of the methods described above.

### BRIEF DESCRIPTION OF DRAWINGS

FIG. 1 shows histological cuts of the dermis and of the epidermis of mice, colored with haematoxylin-eosin.

FIG. 2 is an immunohistochemical analysis of mouse cuts by anti-CD44.

FIG. 3 is a Western blot analysis of protein extracts from mouse skin with an anti-CD44v3 antibody.

FIG. 4 is a Western blot analysis of protein extracts from mouse skin with an 25 kDa anti-pro-HB-EGF antibody.

FIGS. 5 and 6 illustrate histological cuts colored by Van Gieson elastin and by Sirius red.

The invention will now be illustrated in a non-limiting way by the following examples.

### Material and Methods

Skin Atrophy Protocol

Hairless SKH mice received twice daily for 5 days a topical treatment on the back with a steroid (0.05% clobetasol propionate or 0.1% desonide) with or without HA fragments (with a molecular weight comprised between 20 and 500 kDa, and obtained by the method including the treatment steps with ultrasound and filtering as described above). These fragments will be designated in the following examples by HAF. Dermal and epidermal atrophy and the concentration of skin hyaluronate were respectively determined by measuring the dermis-epidermis thickness in optical microscopy and by ELISA.

Inflammation Induced by TPA in the Ear of Mice

Skin inflammation was induced by topical application of 0.005% TPA (12-o-tetradecanoylphorbol-13-acetate) in acetone, on the ears of C57B1/6 mice; the control animals received the same volume of acetone. Clobetasol propionate (0.05%) and the HAFs (1%) were dissolved in 100 μL of carrier, and were applied together with TPA for 4 days; the control animals received the same volume of carrier. The inflammation was determined by measuring the thickness of the ears with a clip and the dermis-epidermis thickness in optical microscopy and by assaying myeloperoxidase activity. The animals were sacrificed 24 hrs after the last application. 6 mm biopsies were sampled, frozen in liquid nitrogen and then stored at −70° C. until the day of the analysis. The remainder of the tissue was set with formol and analyzed by immunohistology.

The myeloperoxidase activity was determined in the supernatant of the homogenates of the ear biopsies. The biopsies, immersed in 1.5 mL of 50 mM sodium phosphate buffer, pH 6.0, containing 0.5% of hexadecyltrimethylammonium bromide (HTAB), were milled for 45 seconds at 0° C. in a Polytron PT 1200 homogenizer. The enzymatic activity of myeloperoxidase was determined according to the method of Bradley et al., modified for using the photometric plate reader. The following reagents were added in wells of 96-well plates: 50 μL of supernatant, 50 μL of phosphate buffer+HTAB, 50 μL of o-dianisidine at 0.68 mg/mL dissolved in water; the reaction was initiated by adding 0.003% hydrogen peroxide prepared extemporaneously. The optical density was measured at 450 nm. The enzymatic activity was compared with that of biopsies of ears only treated with TPA. The expression of CD44, CD44v3 and pro-HB-EGF was analyzed by immunohistochemistry and by Western blotting according to methods already described (PLoS Med 3 (12): e493, 2006).

### Results

The epidermal and cutaneous (distance between the granular layer and the sweating glands) thicknesses were measured by an ocular micrometer. Ten measurements were made per mouse. The results are grouped in Table 1 below. The prevention index is the ratio between the control treated with clobetasol propionate (PC) alone and the PC +HAF composition.

TABLE 1

| | | | Prevention |
|---|---|---|---|
| | PC 0.05% | PC + HAF 1% | index |
| Epidermal | 33 | 248 | 7.5 |
| thickness | | | |
| % of non- | | | |
| treated control | | | |
| Standard | 8 | 42 | |
| deviation | | | |
| Cutaneous | 56 | 84 | 1.5 |
| thickness | | | |
| % of non- | | | |
| treated control | | | |
| Standard | 10 | 17 | |
| deviation | | | |

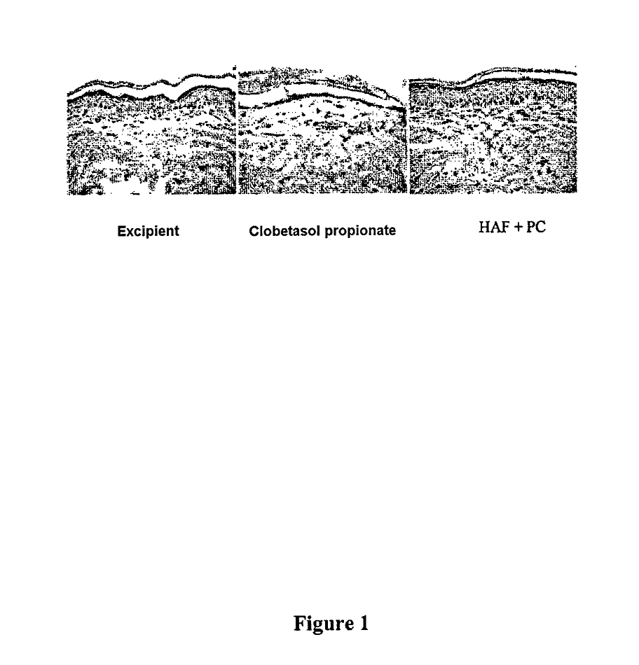

FIG. 1 shows histological cuts of the dermis and of the epidermis of mice, colored with haematoxylin-eosin.

These results demonstrate that the HAFs prevent skin atrophy induced by clobetasol propionate (PC).

The epidermal thickness was measured with an ocular micrometer after treatment with different desonide concentrations. Ten measurements were made per mouse. The results are grouped in Table 2. The prevention index is the ratio, at a determined corticosteroid concentration, between the control treated by the corticosteroid alone and by the corticosteroid composition+HAF.

TABLE 2

| | Desonide % | | | | | | |
|---|---|---|---|---|---|---|---|
| | 0.025% | 0.05% | 0.075% | 0.1% | 0.025% | 0.05% | 0.1% |
| HAF | 0 | 0 | 0 | 0 | 1% | 1% | 1% |
| Epidermal | 72 | 62 | 55 | 46 | 108 | 93 | 78 |
| thickness | | | | | | | |
| % of | | | | | | | |
| non- | | | | | | | |
| treated | | | | | | | |
| control | | | | | | | |
| Standard | 4 | 3 | 4 | 3 | 8 | 9 | 4 |
| deviation | | | | | | | |
| Prevention | | | | | 1.5 | 1.5 | 1.7 |
| index | | | | | | | |

These results therefore demonstrate that HAFs prevent epidermal atrophy induced by desonide in a dose-dependent way.

The epidermal thickness was measured with an ocular micrometer after treatment with different corticosteroids. Ten measurements were made per mouse. The results are grouped in Table 3. The prevention index is the ratio between the control treated by the corticosteroid alone and the corresponding composition comprising the HAFs.

TABLE 3

| | 0.05% Desonide | | Prevention |
|---|---|---|---|
| | (D) | D + HAF | index |
| Epidermal | 62 | 93 | 1.5 |
| thickness | | | |
| % of non- | | | |
| treated | | | |
| control | | | |
| Standard | 3 | 9 | |
| deviation | | | |
| | 0.05% | | |
| | Betamethasone | | Prevention |
| | (B) | B + HAF | index |
| Epidermal | 53 | 116 | 2.2 |
| thickness | | | |
| % of non- | | | |
| treated | | | |
| control | | | |
| Standard | 5 | 12 | |
| deviation | | | |
| | 0.05% Clobetasol | | Prevention |
| | (Cl) | Cl + HAF | index |
| Epidermal | 28 | 207 | 7.4 |
| thickness | | | |
| % of non- | | | |
| treated | | | |
| control | | | |
| Standard | 3 | 16 | |
| deviation | | | |
| | Average of the 3 | | Prevention |
| | steroids (CS) | CS + HAF | index |
| Epidermal | 47 | 139 | 2.9 |
| thickness | | | |
| % of non- | | | |
| treated | | | |
| control | | | |
| Standard | 11 | 36 | |
| deviation | | | |

These results therefore demonstrate that HAFs prevent epidermal atrophy induced by different topical corticosteroids (CS).

The non-treated hyaluronic acid, the fragments obtained by action of hyaluronidase, as well as the HAFs were compared for their preventive effects. The epidermal thickness was measured with an ocular micrometer. Ten measurements were made per mouse. The results are grouped in Table 4. The prevention index is the ratio between the control treated with the corticosteroid alone and with each corticosteroid composition+HAF.

TABLE 4

| | | D + HA | | |
|---|---|---|---|---|
| | Desonide | non- | D + HA tt | |
| | 0.1% (D) | treated | hyaluronidase | D + HAF |
| Epidermal | 46 | 46 | 49 | 78 |
| thickness | | | | |
| % of non- | | | | |
| treated | | | | |
| control | | | | |
| Standard | 3 | 5 | 3 | 4 |
| deviation | | | | |
| Prevention | | 1 | 1.1 | 1.7 |
| index | | | | |
| | 0.05% | C + HA | | |
| | Clobetasol | non- | C + HA tt | |
| | (C) | treated | hyaluronidase | C + HAF |
| Epidermal | 28 | 34 | 31 | 207 |
| thickness | | | | |
| % of non- | | | | |
| treated | | | | |
| control | | | | |
| Standard | 3 | 3 | 5 | 16 |
| deviation | | | | |
| Prevention | | 1.2 | 1.1 | 7.4 |
| index | | | | |

Unlike the HAFs, the fragments prepared by the action of the hyaluronidase, as well as the non-treated hyaluronic acid, do not prevent epidermal atrophy induced by clobetasol propionate or desonide.

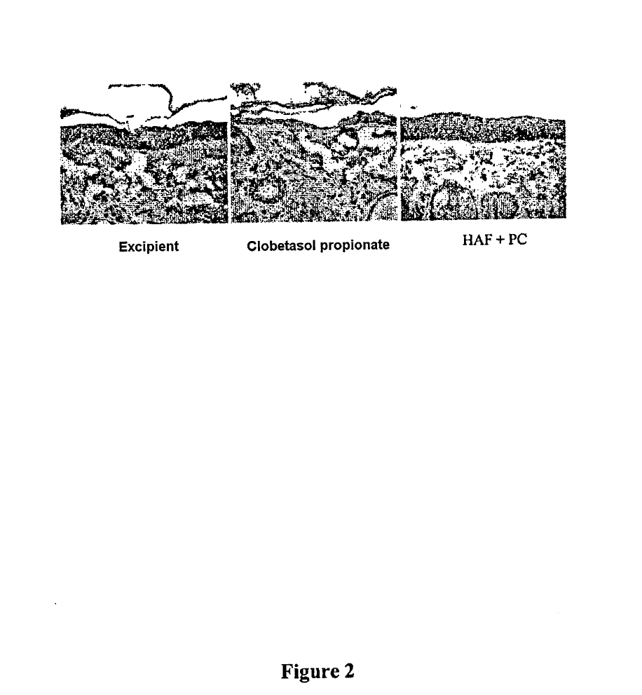

FIG. 2 is an immunohistochemical analysis of mouse cuts by anti-CD44. It shows that the HAFs restore and increase the expression of CD44 in the skin of mice treated with clobetasol propionate.

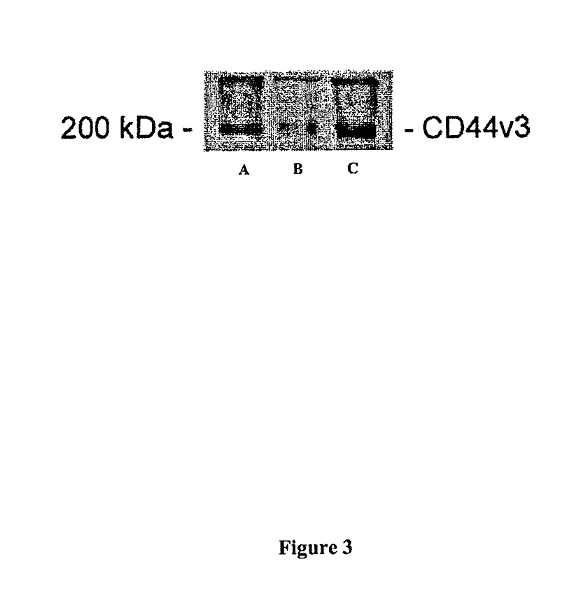

FIG. 3 is a Western blot analysis of protein extracts from mouse skin with an anti-CD44v3 antibody. It shows that the fragments of HAF restore and increase the expression of CD44v3 in the skin of mice treated with desonide and therefore the potentializing effect of the HAFs.

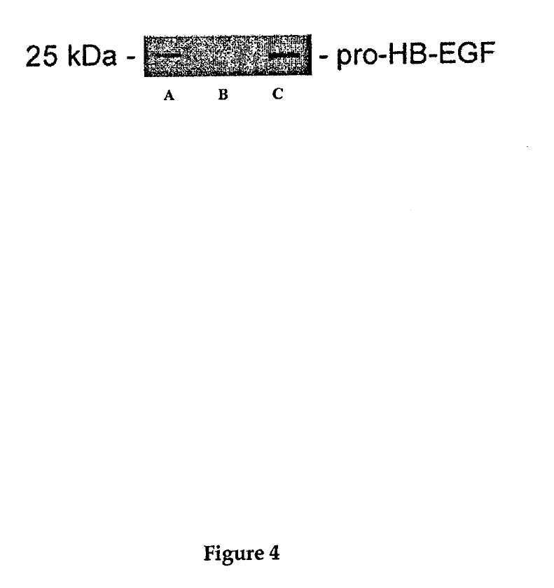

FIG. 4 is a Western blot analysis of protein extracts from mouse skin with an 25 kDa anti-pro-HB-EGF antibody, A representing the carrier, B, clobetasol propionate and C, clobetasol propionate+HAF. It shows that the HAFs restore and increase the expression of pro-HB-EGF in the skin of mice treated with clobetasol propionate.

The epidermal inflammation induced by the application of Phorbol TPA ester was measured, after application of TPA, and then after treatment with clobetasol propionate, and after treatment with the composition comprising clobetasol propionate and HAFs. Ten measurements were made per mouse. The results are grouped in the Table 5 below. The anti-inflammation index is the ratio between the control treated with TPA, and with the composition TPA+PC or TPA+PC+HAF.

TABLE 5

| | | TPA + | TPA + PC + |
|---|---|---|---|
| | TPA | PC | HAF |
| Epidermis inflammation | 438 | 179 | 150 |
| % of control | | | |
| Standard deviation | 38 | 21 | 13 |
| Anti-inflammation index | | 2.4 | 2.9 |

The dermal inflammation induced by applying Phorbol TPA ester was measured, after application of TPA, and then after treatment with clobetasol propionate and after treatment with the composition comprising clobetasol propionate and HAFs. Ten measurements were made per mouse, the results are grouped in the Table 6 below. The anti-inflammation index in the ratio between the control treated with TPA, and with the association TPA+PC or TPA+PC+HAF.

TABLE 6

| | | TPA + | TPA + PC + |
|---|---|---|---|
| | TPA | PC | HAF |
| Dermis inflammation | 226 | 103 | 97 |
| % of non-treated control | | | |
| Standard deviation | 33 | 10 | 8.5 |
| Anti-inflammation index | | 2.2 | 2.3 |

Dermal cellularity induced by applying Phorbol TPA ester was measured, after application of TPA, and then after treatment with clobetasol propionate, and after treatment with the composition comprising clobetasol propionate and HAFs. Ten measurements were made per mouse. The results are grouped in Table 7. The anti-inflammation index is the ratio between the control treated with TPA, and with the association TPA+PC or TPA+PC+HAF.

TABLE 7

| | | TPA + | TPA + PC + |
|---|---|---|---|
| | TPA | PC | HAF |
| Dermal cellularity % of | 789 | 135 | 123 |
| control | | | |
| Standard deviation | 132 | 11 | 9 |
| Anti-inflammation index | | 5.8 | 6.4 |

The cutaneous myeloperoxidase activity induced by applying Phorbol TPA ester was measured, after application of TPA, and then after treatment with clobetasol propionate, and after treatment with the composition comprising clobetasol propionate and HAFs. Ten measurements were made per mouse. The results are grouped in Table 8. The anti-inflammation index is the ratio between the control treated with TPA, and with the association TPA+PC or TPA+PC+HAF.

TABLE 8

| | | TPA + | TPA + PC + |
|---|---|---|---|
| | TPA | PC | HAF |
| myeloperoxidase | 3273 | 38 | 84 |
| % of control | | | |
| Standard deviation | 308 | 8 | 6 |
| Anti-inflammation index | | 39.4 | 39 |

The HAFs do not inhibit the anti-inflammatory effect of clobetasol propionate but on the contrary potentialize the anti-inflammatory effect.

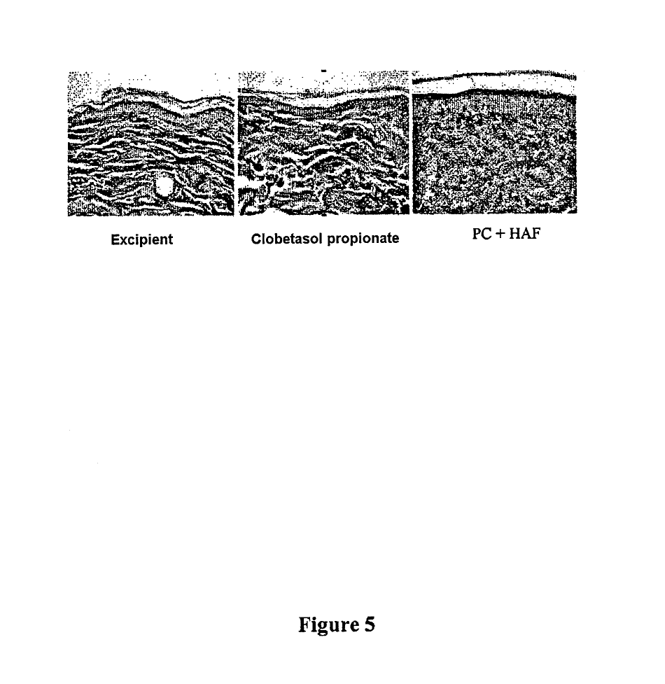

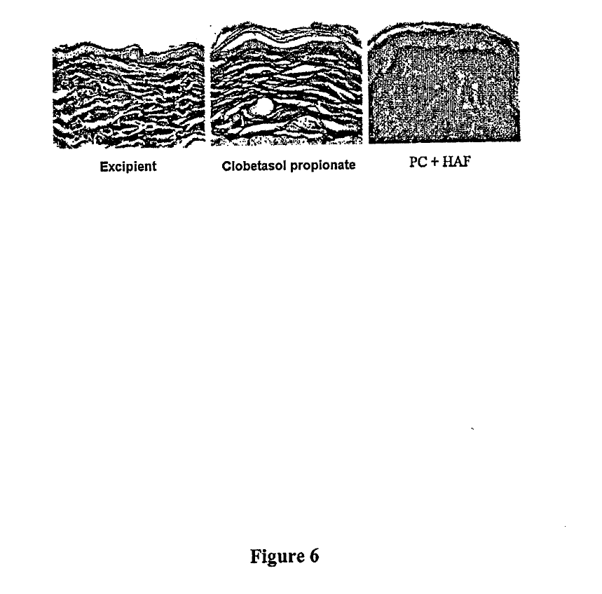

With FIGS. 5 and 6 which illustrate histological cuts colored by Van Gieson elastin and by Sirius red, it was possible to demonstrate that the HAFs protect the elastic network and the dermal collagen from destruction by clobetasol propionate.

1. A method for reducing skin atrophy due to topical administration of corticosteroids comprising:
administering to a person in need thereof a anti-inflammatory dermatological composition intended for topical administration
comprising 0.005 to 0.1% by weight of a corticosteroid and 0.1 to 1% by weight of hyaluronate fragments with an average molecular weight comprised between 20 and 500 kDa.

2. The method according to claim 1, wherein the composition comprises 0.05% by weight of a corticosteroid and 0.5% by weight of hyaluronate fragments.

3. The method according to claim 1, wherein the composition comprises 0.01% by weight of a corticosteroid and 1% by weight of hyaluronate fragments.

4. The method according to claim 1, wherein the corticosteroid is selected from alclometasone dipropionate, amcinonide, beclometasone dipropionate, betamethasone benzoate, betamethasone dipropionate, betamethasone valerate, budesonide, clobetasol propionate, clobetasol butyrate, desonide, desoximethasone, dexamethasone, diflorasone diacetate, diflucortolone valerate, flurandrenolone, fluprednidene acetate, fluocortolone, fluocortine butyl, fluocinonide, fluocinolone acetonide, fluclorolone acetonide, flumetasone pivalate, feudiline hydrochloride, flumetholone, halcinonide, hydrocortisone, hydrocortisone acetate, hydrocortisone butyrate, hydrocortisone valerate, methylprednisolone acetate, mometasone furoate, methylprednisolone, prednisolone, triamcinolone acetonide as well as mixtures thereof.

5. The method according to claim 1, wherein the corticosteroid is clobetasol propionate.

6. The method according to claim 1, wherein the composition further comprises one or more pharmaceutically acceptable excipients.

7. The method according to claim 1, wherein the composition further comprises a pharmaceutically acceptable emollient base.

8. The method according to claim 1, wherein the composition further comprises other dermatologically acceptable excipients for its presentation as a cream, balm, gel, spray, ointment, lotion, film-forming solution, transdermal composition, foam, or shampoo.

9. A method for reducing skin atrophy due to topical administration of corticosteroids, comprising:
administering separately, simultaneously, or spread out in time to a person in need thereof a cream comprising one corticosteroid and another cream comprising hyaluronate fragments of average molecular weight comprised between 20 and 50 kDa.

10. The method according to claim 1, wherein the hyaluronate fragments are obtainable by heat treatment at a temperature above 100° C. of fibers of sodium hyaluronate with high molecular weight, or by treatment with ultrasound of fibers of sodium hyaluronate with high molecular weight, for 10 to 90 minutes, at 400 W and at 4° C., followed by filtering on a gel.

11. The method according to claim 1, wherein the composition comprises 0.01 to 0.05% by weight of a corticosteroid.

12. The method according to claim 1, wherein the composition comprises 0.5 to 1% by weight of hyaluronate fragments.

13. The method according to claim 1, wherein the hyaluronate fragments have an average molecular weight of between 20 and 375 kDa.

14. The method according to claim 1, wherein the hyaluronate fragments have an average molecular weight of between 20 and 150 kDa.

15. The method according to claim 9, wherein the hyaluronate fragments are obtainable by heat treatment at a temperature above 100° C. of fibers of sodium hyaluronate with high molecular weight, or by treatment with ultrasound of fibers of sodium hyaluronate with high molecular weight, for 10 to 90 minutes, at 400 W and at 4° C., followed by filtering on a gel.

16. The method according to claim 9, wherein the hyaluronate fragments have an average molecular weight of between 20 and 375 kDa.

17. The method according to claim 9, wherein the hyaluronate fragments have an average molecular weight of between 20 and 150 kDa.
